(19) United States
(12) Patent Application Publication
(10) Pub. No.: US 2025/0366756 A1
(43) Pub. Date: Dec. 4, 2025

(54) ELECTROCARDIOGRAM DATA DETECTION DEVICE

(71) Applicant: Alps Alpine Co., Ltd., Tokyo (JP)

(72) Inventor: Takahiro KAWAUCHI, Miyagi-ken (JP)

(21) Appl. No.: 19/199,711

(22) Filed: May 6, 2025

(30) Foreign Application Priority Data
May 31, 2024 (JP) 2024-088828

(51) Int. Cl. A61B 5/282; A61B 5/00; A61B 5/26; A61B 5/332

(52) U.S. Cl. CPC A61B 5/282; A61B 5/26; A61B 5/332; A61B 5/6893; A61B 5/702

(57) ABSTRACT

An electrocardiogram data detection device includes a plurality of electrodes provided in a seat of a vehicle to face the human body, a differential signal generation unit that generates a differential signal between signals of two of the plurality of electrodes and generates a plurality of differential signals from the signals of a plurality of pairs of the electrodes, and an electrocardiogram data detection unit that obtains electrocardiogram data based on the differential signals. The electrodes include at least three first electrodes disposed in a width direction of the seat back within a first region of the seat back close to the seat bottom and at least one second electrode within a second region of the seat back located above the first region. The differential signals are at least six differential signals obtained from the signals of the at least three first electrodes and at least one second electrode.

### CLAIM OF PRIORITY

This application claims benefit of Japanese Patent Application No. 2024-088828 filed on May 31, 2024, which is hereby incorporated by reference.

### BACKGROUND OF THE INVENTION

### 1. Field of the Invention

The present disclosure relates to an electrocardiogram data detection device.

### 2. Description of the Related Art

Electrocardiogrameasurement devices have been developed that include a pair of insulated measuring electrodes to be disposed so as to face the human body in an electrically insulated state and a measurement circuit that detects changes in voltage caused by the capacitance coupling between each of the insulated measuring electrodes and the human body, amplifies the difference between the two voltages detected by the two insulated measuring electrodes, and outputs the amplified voltage as an electrocardiogramaveform signal (refer to, for example, Japanese Unexamined Patent Application Publication No. 2007-082938).

### SUMMARY OF THE INVENTION

When an existing electrocardiogrameasurement device is installed on the seat of a vehicle, such as a car, and if the human body moves due to vibration caused by the movement of the vehicle, the distance between the insulated measuring electrode and the human body may change significantly, which may result in failure to measure electrocardiogram data.

Accordingly, the present disclosure provides an electrocardiogram data detection device that can detect electrocardiogram data of a person sitting on a seat, even when the electrocardiogram data detection device is installed in a seat of a vehicle.

According to an embodiment of the present disclosure, an electrocardiogram data detection device includes a plurality of electrodes provided in a seat back of a seat that includes a seat bottom and the seat back and that is installed in a vehicle, where the plurality of electrodes are disposed to face the human body of a person sitting on the seat, a differential signal generation unit configured to generate a differential signal between signals of two of the plurality of electrodes, where the differential signal generation unit generates a plurality of differential signals from the signals of a plurality of pairs of the electrodes, and an electrocardiogram data detection unit configured to obtain electrocardiogram data based on the plurality of differential signals. The plurality of electrodes include at least three first electrodes disposed in the width direction of the seat back within a first region of the seat back close to the seat bottom and at least one second electrode disposed within a second region of the seat back located above the first region, and the plurality of differential signals are at least six differential signals obtained from the signals of the at least three first electrodes and the signal of at least one second electrode.

### BRIEF DESCRIPTION OF THE DRAWINGS

FIG. 1 illustrates an example of the configuration of an electrocardiogram data detection device according to an embodiment;

FIG. 2 illustrates the arrangement of four electrodes in a seat back;

FIG. 3 illustrates an example of the arrangement of the four electrodes as viewed from the front;

FIG. 4 illustrates 12 possible locations where the electrodes are disposed in an experiment;

FIG. 5A illustrates an example of the measurement results of electrocardiogram data;

FIG. 5B illustrates an example of five selectable arrangements of a plurality of electrodes;

FIG. 6A illustrates an example of the measurement results of electrocardiogram data; and

FIG. 6B illustrates an example of the arrangements of the plurality of electrodes according to the embodiment and comparative examples 1 to 4.

### DESCRIPTION OF THE PREFERRED EMBODIMENTS

An electrocardiogram data detection device according to the present disclosure is described below.

### Embodiment

### Electrocardiogram Data Detection Device

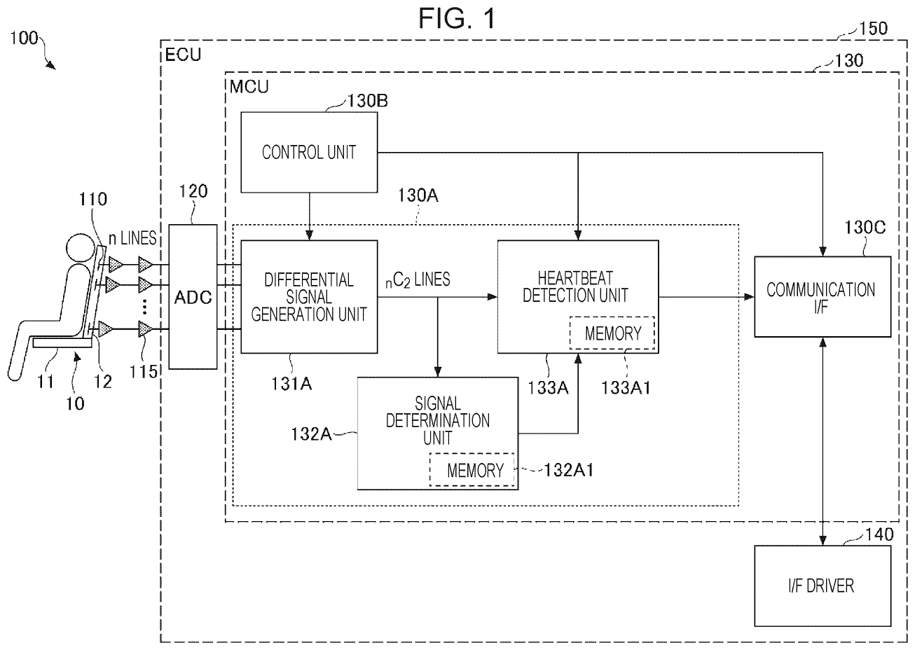

FIG. 1 illustrates an example of the configuration of an electrocardiogram data detection device 100 according to an embodiment. The electrocardiogram data detection device 100 includes an electrode 110, a buffer circuit 115, an analog to digital convertor (ADC) 120, a micro controller unit (MCU) 130, and an interface (I/F) driver 140. The ADC 120, the MCU 130, and the I/F driver 140 constitute an electronic control unit (ECU) 150. The ECU 150 is mounted in, for example, a vehicle and is connected to a higher-layer device via, for example, an in-vehicle network.

The electrocardiogram data detected by the electrocardiogram data detection device 100 is data indicating the electrical activity of the heart. The electrocardiogram data includes data including electrocardiogram waveforms or the like or heartbeat data representing the heartbeat (heartbeat interval, or heart rate obtained by the heartbeat interval) obtained based on the electrocardiogram data. According to the present embodiment, an example in which the heartbeat interval is obtained is described.

An example of the electrocardiogram data detection device 100 is a device for detecting electrocardiogram data of a driver sitting on the driver's seat 10 of a car. The car is an example of the vehicle. The seat 10 includes a seat bottom 11 and a seat back 12. FIG. 1 illustrates the seat 10 for the driver of a car. However, the electrocardiogram data detection device 100 may be a device for detecting electrocardiogram data of a passenger (not the driver) sitting on a seat 10 other than the driver's seat 10 of the car. The vehicle is not limited to a car, but may be a truck, a bus, a cab, a train, a ship, an aircraft, or the like. Of course, an automobile and an electric vehicle are included in vehicles.

Hereinafter, the description is made with reference to the left, right, forward, backward, up, and down of a car having the seat 10 installed therein. Forward is the direction in which the car moves forward, and backward is the direction in which the car moves backward. Left represents the left-hand side when looking in the forward direction, while right represents the right-hand side also when looking in the forward direction. Up and down are vertical directions. The seat 10 is mounted in the interior of the car to face the front of the car. Therefore, left, right, frontward, backward, up, and down of the human body of the person sitting on the seat 10 are the same as the above-described left, right, frontward, backward, up, and down of the car.

### Electrodes

A plurality of electrodes 110 are provided inside the seat back 12 of the seat 10 for the driver of the car. More specifically, the plurality of electrodes 110 are provided on the backside of the surface skin of the seat back 12 and face the waist or back of the driver sitting on the seat 10. The surface skin of the seat back 12 is the outer surface of the seat back 12 that is in contact with the waist or back of the seated driver.

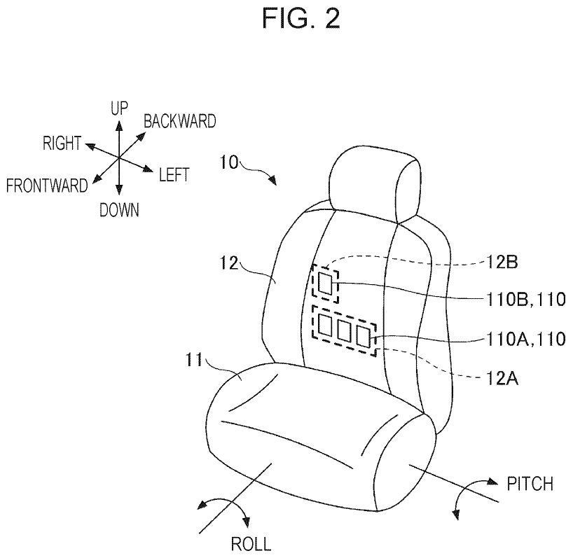

Hereinafter, the description is made with reference to, as an example, the configuration in which four electrodes 110 are arranged in two blocks in the up-down direction. More specifically, three of the electrodes 110 are arranged in the lower block, and one of the electrodes 110 is arranged in the upper block. The sizes of the four electrodes 110 are the same, for example. The arrangement of the four electrodes 110 are described in more detail below with reference to FIG. 2. However, the number of electrodes 110 is not limited to four. It is desirable that the number of electrodes 110 be at least four, at least three of which are arranged in the lower block and at least one is arranged in the upper block. The details are discussed below.

The electrodes 110 are capacitively coupled to the body of the driver that the electrodes 110 face via the surface skin or clothing of the seat 10 to measure the electrical signals generated in the human body during heart activity (beating) as induced signals generated in the electrodes 110. Therefore, by detecting the voltage difference between two of the four electrodes 110, the potential difference between two points on the body can be detected, and the electrocardiogram data can be measured. Each of the electrodes 110 is connected to the ECU 150 via a buffer circuit 115, such as a voltage follower circuit. The voltage signal input from the buffer circuit 115 to the ECU 150 represents the voltage of each of the electrodes 110.

### ADC

The ADC 120 is provided between the buffer circuit 115 and the MCU 130. The ADC 120 converts the voltage signal input from the buffer circuit 115 into a digital signal by sampling the voltage signal at sufficiently short time intervals, such as several milliseconds, and outputs the digital signal to the MCU 130.

According to the present embodiment, the ADC 120 is always operating and outputting signals to the electrocardiogram data detection device 100. However, the ADC 120 may be operated intermittently to perform conversions for 60 seconds or longer.

### MCU

The MCU 130 includes an arithmetic unit 130A, a control unit 130B, and a communication I/F 130C. As the MCU 130, a computer is used that includes a central processing unit (CPU), a random-access memory (RAM), a read only memory (ROM), an input/output interface, and an internal bus.

The arithmetic unit 130A includes s a differential signal generation unit 131A, a signal determination unit 132A, and a heartbeat detection unit 133A. The signal determination unit 132A and the heartbeat detection unit 133A include a memory 132A1 and a memory 133A1, respectively. The heartbeat detection unit 133A is an example of an electrocardiogram data detection unit.

The arithmetic unit 130A, the control unit 130B, and communication I/F 130C, as well as the differential signal generation unit 131A, the signal determination unit 132A, and the heartbeat detection unit 133A inside of the arithmetic unit 130A, represent the functions of a program executed by the MCU 130 in the form of functional blocks. The memories 132A1 and 133A1 are functional representations of the memory of the MCU 130. The MCU 130 may include a memory other than the memories illustrated herein, but the memory is not illustrated in FIG. 1.

### Arithmetic Unit

The arithmetic unit 130A generates heartbeat data, such as heartbeat intervals, based on the voltage values output from the ADC 120 and outputs the generated heartbeat data to the communication I/F 130C.

### Differential Signal Generation Unit

The differential signal generation unit 131A selects two of the four voltage values output from the ADC 120 and calculates a differential signal at all times. In the subtraction, there are four voltage values as the subtrahends and four voltage values as the minuends. Therefore, if all the differential signals are simply calculated, 16(=4×4) differential signals can be obtained. However, the difference value calculated based on the same electrode is zero. For this reason, there is no point in computing the differential signal. In addition, when the subtrahend and the minuend are switched, only the sign of the difference value obtained by calculation is opposite to that before the switching and, thus, the two electrocardiogram data can be regarded as the same. Therefore, there is no point in calculating the differential signals both before and after the switching. For this reason, according to the present embodiment, six (=4C2 =(4*3)/2) differential signals are generated by selecting two different voltages from the four voltage values. Each of the differential signals is identified by a combination of the two electrodes 110. The differential signals are evaluated by the ratio of the signal level to the noise level (S/N ratio) in the signal determination unit 132A. The differential signal generation unit 131A outputs the differential signals each associated with the electrodes that measured the differential signal to the signal determination unit 132A and the heartbeat detection unit 133A.

### Signal Determination Unit

The signal determination unit 132A obtains the S/N ratio of each of the six differential signals input from the differential signal generation unit 131A, determines whether the differential signal is suitable for detecting electrocardiogram data (for example, determines whether the S/N ratio of the differential signal is higher than or equal to a predetermined S/N ratio), and outputs the determination result to the heartbeat detection unit 133A.

The S/N ratio obtained by the signal determination unit 132A is the average of the values obtained within a predetermined time period (for example, 60 seconds). When the S/N ratio is obtained continuously, the differential signal data for 60 seconds output from the ADC 120 and the differential signal data for 60 seconds for obtaining the next S/N ratio may be continuous data or data with a predetermined time lag therebetween. Alternatively, the differential signal data may partially overlap. For example, the first S/N ratio may be obtained by calculation based on the differential signal output from the ADC 120 from time 0 seconds to time 60 seconds, and the second S/N ratio may be obtained by calculation based on the differential signal output from the ADC 120 from time 30 seconds to time 90 seconds.

As an example, the determination result of the signal determination unit 132A represents data that are selected from the six differential signals and that have a predetermined S/N ratio or higher, ordered from the highest to the lowest S/N ratio, and the electrode numbers corresponding to the S/N ratio. Another example of the determination result indicates that there is no data with a predetermined S/N ratio or higher. The calculation logic for determining the S/N ratio in the signal determination unit 132A is stored in the memory 132A1, and the determination result of the signal determination unit 132A is also stored in the memory 132A1.

### Heartbeat Detection Unit

The heartbeat detection unit 133A stores, in the memory 133A1, the differential signals input from the differential signal generation unit 131A. For the differential signal that is determined by the signal determination unit 132A to be suitable for detecting electrocardiogram data, the heartbeat detection unit 133A records the time when a heartbeat (cardiac beat) occurs based on the differential signal input from the differential signal generation unit 131A. If the time when the immediately previous heartbeat occurs has been recorded, the heartbeat detection unit 133A records the time interval from the immediately previous heartbeat.

The electrocardiogram data is based on electrical signals produced by the contraction of the atria and ventricles of the heart and includes the P wave, QRS wave, T wave, and the like. The time when a heartbeat occurs in each of the differential signals is identified by the peak position of the QRS wave, which is the largest signal. In reality, however, the waveform of the measured electrocardiogram data varies with the mounted position of the electrode relative to the heart, so that the time when a heartbeat occurs is identified based on the predetermined calculation logic stored in the memory 133A1.

The heartbeat detection unit 133A outputs, to the communication I/F 130C, the time when a heartbeat occurs or heartbeat data that represents a time interval and that corresponds to a differential signal. The heartbeat data corresponding to one differential signal consists of a plurality of data corresponding to the number of heartbeats in 60 seconds.

The communication I/F 130C transmits the heartbeat data input from the heartbeat detection unit 133A to the higher-layer device of the ECU 150 via the in-vehicle network driven by the I/F driver 140. The heartbeat data is then used, for example, to evaluate the driver's level of tension, stress, or drowsiness.

### Arrangement of Electrodes

FIG. 2 illustrates the arrangement of the four electrodes 110 in the seat back 12. The seat back 12 has a first region 12A and a second region 12B. The first region 12A is located on the lower side, and the second region 12B is located on the upper side than the first region 12A.

The four electrodes 110 are divided into three first electrodes 110A provided within the first region 12A and one second electrode 110B provided within the second region 12B. The three first electrodes 110A are arranged horizontally, parallel to each other, within the first region 12A. The second electrode 110B is located above the three first electrodes 110A. For example, the second electrode 110B is located at the same position in the left-right direction as the rightmost first electrode 110A out of the three first electrodes 110A. The second region 12B having the one second electrode 110B disposed therein is smaller than the first region 12A having the three first electrodes 110A disposed therein, and the center of the second region 12B is located to the right of the center of the first region 12A in the left-right direction. Hereinafter, when the distinction between the first electrode 110A and the second electrode 110B is not needed, the first electrode 110A and the second electrode 110B are collectively referred to as “electrodes 110”.

Vibration caused by car driving causes the seat 10 to vibrate in the roll direction and in the pitch direction. When the vibrations occur, the driver's upper body swings, causing the upper body to easily shift from the seat back 12.

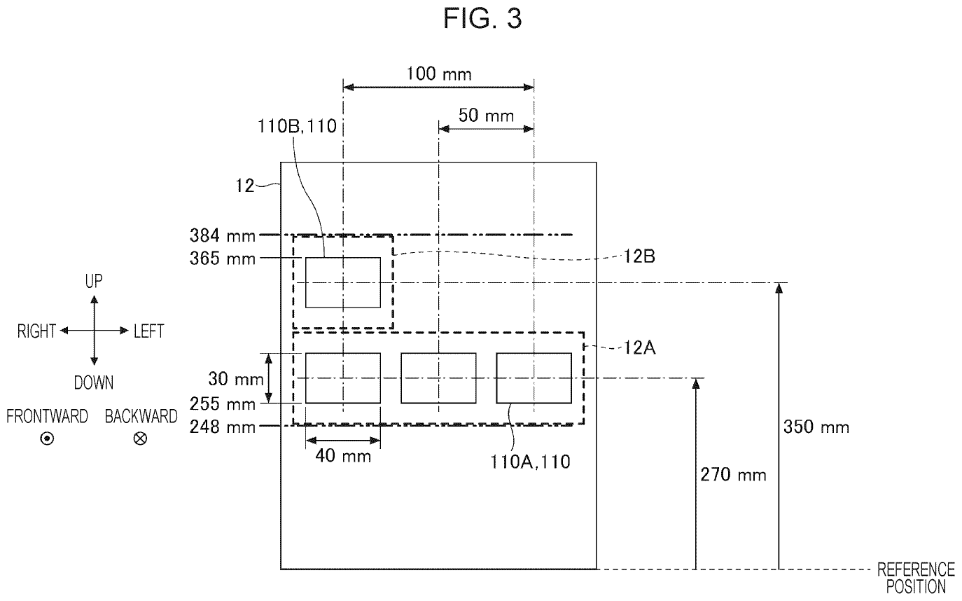

The arrangement of the four electrodes 110 is described below with reference to FIG. 3 in addition to FIG. 2. FIG. 3 illustrates an example of the arrangement of the four electrodes 110 as viewed from the front. FIG. 3 is a transparent view of the arrangement of the four electrodes 110 when the seat back 12 is viewed from the front. FIG. 3 also illustrates the first region 12A and the second region 12B. As used herein, the term “viewing from the front” refers to viewing the front surface of an object from the front side to the back side.

The second region 12B is smaller than the first region 12A and is located above the first region 12A. The center of the second region 12B is located to the right of the first region 12A in the left-right direction. Therefore, the center of the second region 12B is located to the upper right of the center of the first region 12A.

The lower end of the seat back 12 illustrated in FIG. 3 is taken as the reference position for the height positions of the first electrodes 110A and the second electrode 110B. It is assumed that the height position of the lower end of the seat back 12 is the same as the height position of the back end of the seating surface of the seat bottom 11.

The seat back 12 is usually set at an angle so as to be slightly tilted backward from a line perpendicular to the horizontal plane. However, as an example, FIG. 3 illustrates the configuration in which the seat back 12 is standing upright as viewed from the front.

The height positions of the first electrodes 110A and the second electrode 110B (270 mm and 350 mm, respectively) illustrated in FIG. 3 indicate the heights from the reference position. The height positions of the first electrodes 110A and the second electrode 110B (270 mm and 350 mm, respectively) also indicate the vertical distance from the reference position when the seat back 12 is standing upright. Thus, when the seat back 12 is tilted backward from a line perpendicular to the horizontal plane, the first electrodes 110A and the second electrode 110B are located at the distances represented by the height positions from the back end of the seating surface of the seat bottom 11 along the direction of the tilt angle.

The three first electrodes 110A and one second electrode 110B are equal in size as viewed from the front. For example, the length in the transverse direction (left-right direction) is 40 mm, and the length in the longitudinal direction (up-down direction) is 30 mm. For example, each of the three first electrodes 110A and one second electrode 110B is rectangular in shape.

The height positions of the three first electrodes 110A are the same, and the pitch of the three first electrodes 110A in the transverse direction is 50 mm. For example, the position of the second electrode 110B in the transverse direction (left-right direction) is the same as the position of the rightmost first electrode 110A out of the three first electrodes 110A.

The reason why the second electrode 110B in the second region 12B is positioned to the right of the center of the human body in the left-right direction is to obtain a differential signal with a higher S/N ratio when measuring electrocardiogram data. In the case of an average person, the vector of electromotive force generated by the beating heart is generally in the direction from the right shoulder to the left leg. Therefore, by setting the electrodes along the electromotive force vector and, more specifically, by positioning the second electrode 110B in the upper second region 12B to the right of the center of the human body in the left-right direction, a stronger differential signal can be obtained.

The electrocardiogram data detection device 100 needs to include at least three first electrodes 110A. For this purpose, four or more first electrodes 110A may be arranged horizontally, parallel to each other, within the first region 12A.

The electrocardiogram data detection device 100 needs to include at least one second electrode 110B. For this purpose, two or more second electrodes 110B may be arranged horizontally, parallel to each other, within the second region 12B. The number of second electrodes 110B needs to be less than the number of first electrodes 110A. If a plurality of second electrodes 110B are provided, the pitch of the plurality of second electrodes 110B in the left-right direction may be equal to the pitch of the plurality of first electrodes 110A.

One or more second electrodes 110B, which are fewer in number than the plurality of first electrodes 110A, are positioned to the right of the plurality of first electrodes 110A in the left-right direction. Therefore, even when a plurality of second electrodes 110B are provided, the center of the second region 12B as viewed from the front is located to the upper right relative to the center of the first region 12A.

The plurality of first electrodes 110A may be arranged in the first region 12A, not parallel to each other in the horizontal direction. In this a case, all of the plurality of first electrodes 110A need to have overlapping sections in the up-down direction. That is, the plurality of first electrodes 110A only need to be arranged along the horizontal direction within the first region 12A.

Similarly, if a plurality of second electrodes 110B are provided, the plurality of second electrodes 110B may be arranged in the second region 12B, not parallel to each other in the horizontal direction. In this a case, all of the plurality of second electrodes 110B need to have overlapping sections in the up-down direction. That is, the plurality of second electrodes 110B only need to be arranged along the horizontal direction within the second region 12B.

For example, the first electrodes 110A and the second electrode 110B are rectangular in shape, but may be other than rectangular in shape. The plurality of first electrodes 110A may have different shapes from each other. Similarly, if a plurality of second electrodes 110B are provided, the plurality of second electrodes 110B may have different shapes from each other.

### Height Positions of Three First Electrodes

For example, the three first electrodes 110A are disposed such that the lower ends thereof are at a height of 255 mm from the reference position. The three first electrodes 110A needs to be disposed above the height position corresponding to the height position of the iliac crest of a driver sitting on the seat 10. The height of the iliac crest of approximately 95% or more of people is 248 mm from the reference position, according to “Makiko Kouchi and Masaaki Mochimaru, 2005: AIST Anthropometric Database, National Institute of Advanced Industrial Science and Technology, H16PRO 287”. That is, the three first electrodes 110A need to be disposed such that the lower ends thereof are disposed at a height of 248 mm or more from the reference position. However, since the second electrode 110B is located above the three first electrodes 110A and, in addition, there is a restriction on the height position of the second electrode 110B, the three first electrodes 110A need to be disposed such that the lower ends thereof are at a height of 248 mm or more from the reference position under the condition that the relationship with the restriction on the height position of the second electrode 110B is satisfied.

### Height Position of Second Electrode

For example, the second electrode 110B is disposed such that the upper end thereof is at a height of 365 mm from the reference position. The second electrode 110B needs to be disposed below the height position corresponding to the height position of the inferior angle of the scapula of a driver sitting on the seat 10. The height position of the inferior angle of the scapula of approximately 95% or more of people is at a height of 384 mm from the reference position, according to “Makiko Kouchi and Masaaki Mochimaru, 2005: AIST Anthropometric Database, National Institute of Advanced Industrial Science and Technology, H16PRO 287”. That is, the second electrode 110B needs to be disposed such that the upper end thereof is at a height of 384 mm or less from the reference position. However, since the three first electrodes 110A are located below the second electrode 110B and, in addition, there is the above-described condition for the lower limit of the lower ends of the first electrodes 110A, the second electrode 110B needs to be disposed such that the upper end thereof is at a height of 384 mm or less under the condition that the relationship with the restriction on the height position of the first electrodes 110A is satisfied.

### Experiment

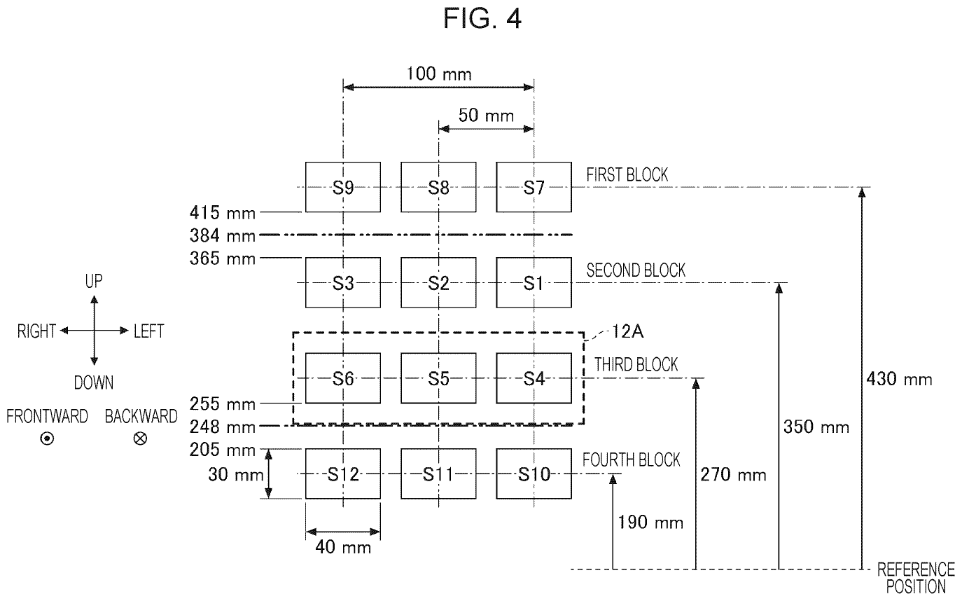

FIG. 4 illustrates 12 regions each allowing one of the electrodes 110 to be disposed therein for the experiment. FIG. 4 illustrates 12 regions S1 to S12 where the electrodes 110 can be disposed in four blocks (four rows) in the up-down direction and three columns in the left- right direction. Each of the sizes of the regions S1 to S12 is equal to the size of the electrode 110 and has, for example, a length of 40 mm in the transverse direction (left-right direction) and a length of 30 mm in the longitudinal direction (up-down direction). FIG. 4 illustrates a first region 12A that is the same as illustrated in FIG. 3, and a second region 12B is not illustrated.

The allocation of the regions S1 to S12 is illustrated in FIG. 4. The first block (uppermost block) consists of regions S7, S8, and S9, from left to right. The second block consists of the regions S1, S2, and S3, from left to right. The third block consists of the regions S4, S5, and S6, from left to right. The lowermost fourth block consists of the regions S10, S11, and S12, from left to right. Each of the regions SI to S12 is a region of the seat back 12 where the electrode 110 can be disposed.

The height of the center of the uppermost first block (S7, S8, and S9) is 430 mm, and the height of the lower end of the first block is 415 mm. Thus, the first block is higher than the height position of the inferior angle of the scapula (384 mm).

The height of the center of the second block (S1, S2, and S3) is 350 mm, and the height of the upper end of the second block is 365 mm. Thus, the second block is lower than the height position of the inferior angle of the scapula. The height position of the second block is equal to the height position of the second electrode 110B illustrated in FIG. 3. The position of the second electrode 110B illustrated in FIG. 3 is the position of the region S3.

The height of the center of the third block (S4, S5, and S6) is 270 mm, and the height of the lower end of the third block is 255 mm. Thus, the third block is higher than the height position of the iliac crest (248 mm). The height position of the third block is equal to the height position of the three first electrodes 110A illustrated in FIG. 3. The regions where the three first electrodes 110A are disposed in FIG. 3 are the regions S4, S5, and S6.

The height of the center of the lowermost fourth block (S10, S11, and S12) is 190 mm, and the height of the upper end of the fourth block is 205 mm. Thus, the fourth block is lower than the height position of the iliac crest (248 mm).

### Electrocardiogram Data Measurement Results

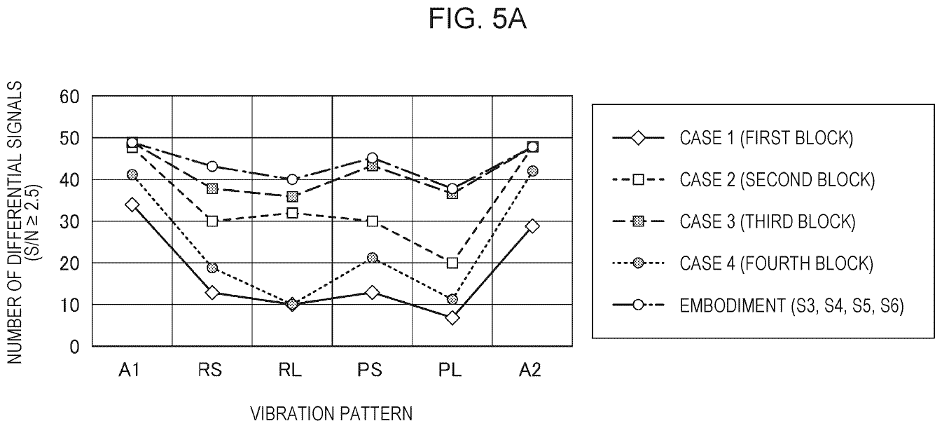

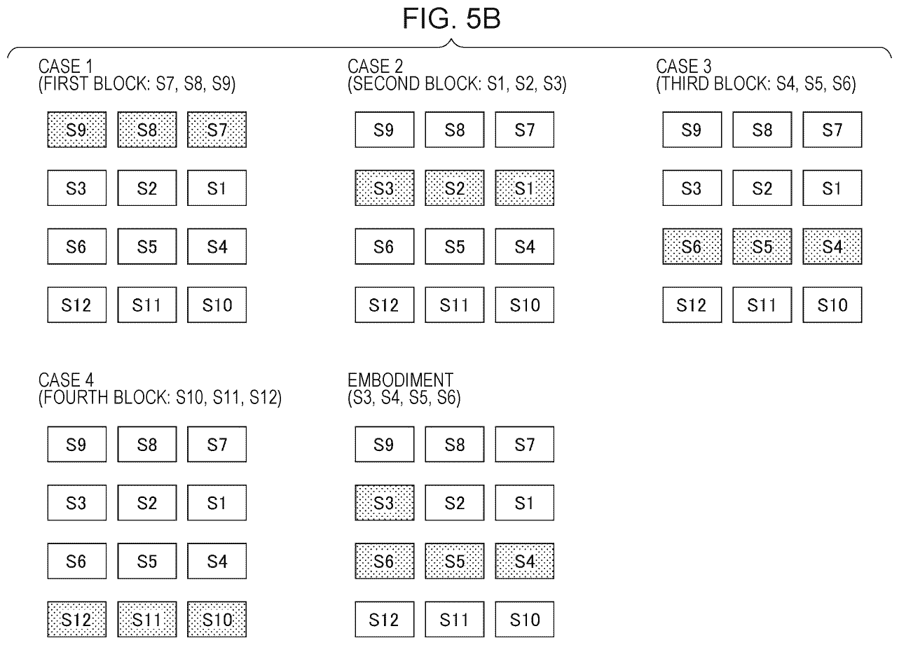

FIG. 5A illustrates an example of the electrocardiogram data measurement results. The seat 10 was mounted on a shake table, and a plurality of electrodes 110 were disposed by selecting regions from the regions S1 to S12 illustrated in FIG. 4 in five different ways, and six different vibration patterns (A1, RS, RL, PS, PL, A2) are generated to measure the electrocardiogram data regarding a seated person.

In FIG. 5A, the abscissa represents the time, and the vibration pattern is changed from A1 to RS to RL to PS to PL to A2 as time passes. The ordinate represents the number of differential signals that had a signal-to-noise (S/N) ratio of 2.5 or higher out of 50 differential signals calculated by the differential signal generation unit 131A through 50 measurements.

The 50 measurements were conducted as described below. Each of 25 adults (18 males and 7 females) was sat on the seat 10, and the measurement was conducted twice per person. In each of the measurements, the differential signal was measured over 60 seconds for each of the vibration patterns. The differential signal with an S/N ratio of 2.5 or higher means that the differential signal is good enough to allow calculation of electrocardiogram data.

As the S/N ratio, the maximum value out of the S/N ratios of a plurality of differential signals obtained from the plurality of electrodes in each measurement was used. This is because if even one good differential signal having an S/N ratio of 2.5 or higher is obtained, the electrocardiogram data can be obtained. For example, if, among the S/N ratios of six differential signals obtained from four electrodes 110, the maximum value was 5.5, the S/N ratio obtained in the measurement is determined to be 5.5. Under such a rule, the number of differential signals with an S/N ratio of 2.5 or higher was measured.

FIG. 5B illustrates an example of five different selections of the arrangement of the plurality of electrodes 110. The five selections include Cases 1 to 4 and Embodiment. In Case 1, three electrodes 110 were disposed in the three regions (S7, S8, S9) in the first block. In Case 2, three electrodes 110 were disposed in the three regions (S1, S2, S3) in the second block. In Case 3, three electrodes 110 were disposed in the three regions (S4, S5, S6) in the third block. In case 4, three electrodes 110 were disposed in three regions (S10, S11, S12) in the fourth block. In Embodiment, like the four electrodes 110 illustrated in FIG. 3, four electrodes 110 were disposed in the region S3 of the second block and in three regions (S4, S5, S6) of the third block. As described above, the regions were selected in five ways to measure the electrocardiogram data.

In the vibration pattern A1, the seat 10 was in a stationary state before being vibrated on the shake table and, thus, the seat 10 was not vibrating. The vibration pattern RS was a vibration pattern in which the shake table produced small vibrations in the roll direction on the seat 10. The vibration pattern RL was a vibration pattern in which the shake table produced large vibrations in the roll direction on the seat 10. The vibration pattern PS was a vibration pattern in which the shake table produced small vibrations in the pitch direction on the seat 10. The vibration pattern PL is a vibration pattern in which the shake table produced large vibrations in the pitch direction on the seat 10. The vibration pattern A2 is a vibration pattern in which the shake table was in a stationary state after the vibration pattern PL and, thus, the seat 10 was not vibrating.

As illustrated in FIG. 5A, the number of differential signals with an S/N ratio of 2.5 or higher in each of Case 1 (first block) and Case 4 (fourth block) was significantly smaller than in each of Case 2 (second block) and Case 3 (third block). Even in the vibration patterns A1 and A2 in which the seat 10 was not vibrating, the number of differential signals with an S/N ratio of 2.5 or higher in Case 1 (first block) was about 30, and the number of differential signals with an S/N ratio of 2.5 or higher in Case 4 (fourth block) was 41 to 42.

In Embodiment, the number of differential signals with an S/N ratio of 2.5 or higher was larger than in Cases 1 to 4 for all the vibration patterns. In Case 3 (third block), all the measurement results were close to those in Embodiment. In Case 2 (second block), the number of differential signals with an S/N ratio of 2.5 or higher was smaller than in Case 3 (third block).

The above-described measurement results suggested that placing the electrodes 110 in the first or fourth blocks did not lead to acquisition of good electrocardiogram data and, thus, measurements for the second and third blocks were conducted as described below.

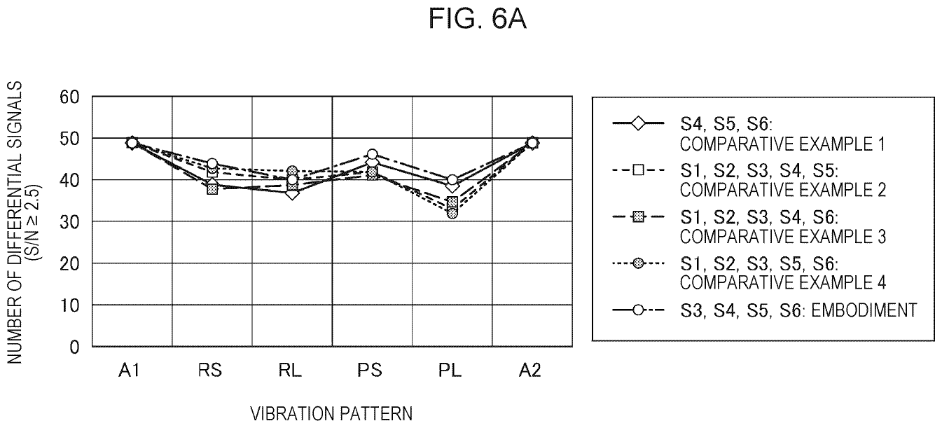

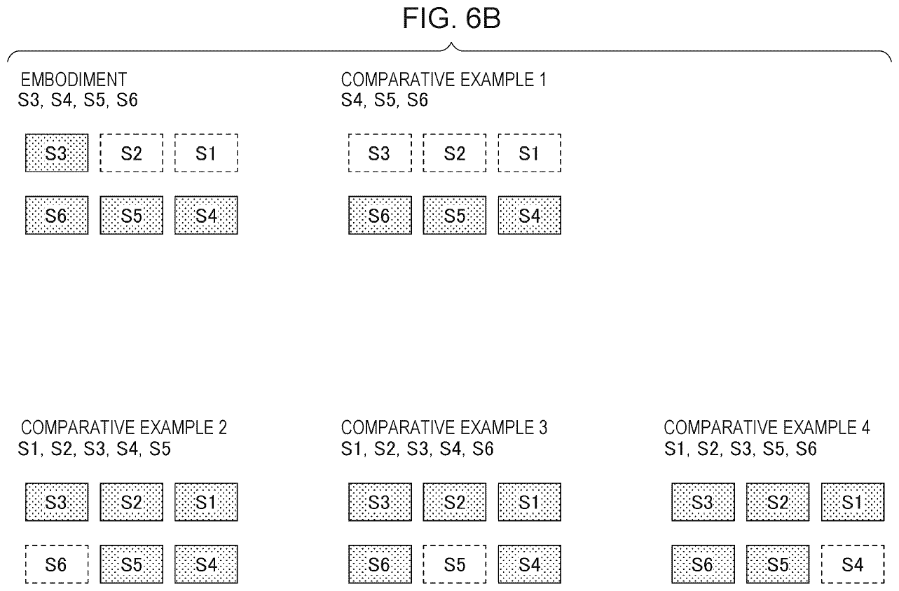

FIG. 6A illustrates an example of the electrocardiogram data measurement results. The seat 10 was set on a shake table, and a plurality of electrodes 110 were disposed by selecting regions from among the regions S1 to S6 illustrated in FIG. 4 in five different ways, and six different vibration patterns (A1, RS, RL, PS, PL, A2) were generated to measure the electrocardiogram data of a seated person. The six vibration patterns are the same as the six vibration patterns described above.

As in FIG. 5A, in FIG. 6A, the abscissa represents the time, and the vibration patterns are changed from A1 to RS to RL to PS to PL to A2 as time passes. The ordinate represents the number of differential signals that had a signal-to-noise (SN) ratio of 2.5 or higher out of 50 electrocardiogram data obtained through 50 measurements. The method for conducting 50 measurements was the same as described above.

The regions were selected from among the regions S1 to S12 in five different ways for Embodiment and Comparative Examples 1 to 4 as described below. FIG. 6B illustrates an example of the arrangements of the plurality of electrodes 110 in Embodiment and in Comparative Examples 1 to 4.

In Embodiment, as for the four electrodes 110 illustrated in FIG. 3, the regions S3, S4, S5, and S6 were selected. In Comparative Example 1, three regions S4, S5, and S6 were selected. In Comparative Example 2, five regions S1, S2, S3, S4, and S5 were selected. In Comparative Example 3, five regions S1, S2, S3, S4, and S6 were selected. In Comparative Example 4, five regions S1, S2, S3, S5, and S6 were selected.

For the vibration patterns A1 and A2 in which the seat 10 did not vibrate, the number of differential signals with an S/N ratio of 2.5 or higher was about 50 in all of Embodiment and Comparative Examples 1 to 4.

The measurement results of the embodiment for vibration patterns RS and RL were particularly better than the measurement results of Comparative Example 1. Addition of an electrode 110 (first electrode 110A) into the region S3 improved the S/N ratio of the electrocardiogram data in terms of roll direction vibration. In contrast, in terms of the pitch direction vibration patterns PS and PL, the measurement result of Embodiment was the same as that of Comparative Example 1, indicating that addition of an electrode 110 (first electrode 110A) into the region S3 was less effective against pitch direction vibration.

In Comparative Examples 2 to 4, of the three regions in the third block, the region where the electrode 110 was not disposed was different from one another. In Comparative Examples 2 to 4, the S/N ratio became deteriorated due to the pitch direction vibration, and the placement of the three electrodes 110 in the three regions in the second block did not produce good results.

As described above, it was found that in terms of pitch direction vibration, Embodiment and Comparative Example 1 in which three electrodes 110 are disposed in the third block provide a better S/N ratio than Comparative Examples 2 to 4. That is, it is found that it is desirable to have at least three electrodes 110 in the third block. In terms of the roll direction vibration, the S/N ratio of Embodiment in which an additional electrode 110 was disposed in the region S3 of the second block was better than that of comparative example 1. That is, it is found that it is desirable to have at least one electrode 110 in the second block and three electrodes 110 in the third block.

In addition to the results of Embodiment, the results of Comparative Example 1 were relatively good, while the results of Comparative Examples 2 to 4 were not. In Comparative Example 1, three electrodes 110 were disposed in the third block and, therefore, three differential signals were obtained from the third block. However, in Comparative Examples 2 to 4, only two electrodes 110 were disposed in the third block and, therefore, only one differential signal was obtained from the third block.

Thus, the possibility exists that the difference in result between Comparative Example 1 and Comparative Examples 2 to 4 is caused by whether three electrodes 110 are disposed in the third block. In the arrangements of electrodes 110 in Comparative Examples 2 to 4, if one more electrode 110 was reduced from the second block, the number of differential signals with an S/N ratio of 2.5 or higher decreased more. More specifically, when, in Comparative Example 2, the electrode 110 in the region S3 was removed, when, in Comparative Example 3, the electrode 110 in the region S2 was removed, and when, in Comparative Example 4, the electrode 110 in the region S1 was removed, the number of differential signals with an S/N ratio of 2.5 or higher remained the same or decreased.

In Embodiment, the possibility exists that an increase in the number of good differential signals with S/N ratio of 2.5 or higher is caused by six differential signals, that is, three differential signals obtained from the regions S4 and S5, from the regions S4 and S6, and from the regions S5 and S6 in the third block, and three differential signals obtained from the region S3 in the second block and each of the regions S4, S5, and S6 in the third block.

The measurement results have been described above with reference to the electrocardiogram data detection device 100 of the embodiment having a configuration including three first electrodes 110A and one second electrode 110B. However, the electrocardiogram data detection device 100 having a configuration including at least four first electrodes 110A or at least two second electrodes 110B provided similar measurement results. Even when the number of first electrodes 110A was increased to five or more, there was no change in the acquisition rate of differential signals with an S/N ratio of 2.5 or higher. For this reason, the upper limit for the number of first electrodes 110A can be set to five.

### Effects

The electrocardiogram data detection device 100 includes the plurality of electrodes 110 provided in the seat back 12 of the seat 10 that includes the seat bottom 11 and the seat back 12 and that is installed in a vehicle, where the plurality of electrodes 110 are disposed facing the human body of a person sitting on the seat 10, the differential signal generation unit 131A that generates a differential signal between signals of two of the plurality of electrodes 110, where the differential signal generation unit 131A generates a plurality of the differential signals from the signals of a plurality of pairs of the electrodes 110, and the heartbeat detection unit 133A (an electrocardiogram data detection unit) that obtains electrocardiogram data based on the plurality of differential signals. The electrodes 110 includes at least three first electrodes 110A disposed in the width direction of the seat back 12 within the first region 12A of the seat back 12 close to the seat bottom 11 and at least one second electrode 110B disposed within the second region 12B of the seat back 12 located above the first region 12A. The plurality of differential signals are at least six differential signals obtained from the signals of the at least three first electrodes 110A and the signal of at least one second electrode 110B.

When the seat is shaken due to the movement of the vehicle, the lower part of the upper body, such as the waist, moves less, but the upper part, such as the shoulder, moves more and tends to move away from the seat back 12. Therefore, even if the seat is shaken due to the movement of the vehicle, at least three first electrodes 110A in the lower first region 12A can acquire at least three signals from the waist portion, which moves relatively little. Since the human body is less likely to be moved away from the at least three first electrodes 110A, at least one differential signal with a high S/N ratio can be easily acquired from the at least three signals obtained from the at least three first electrodes 110A. In addition, even if the seat is shaken, the at least one second electrode 110B in the upper second region 12B acquires a signal. Therefore, at least one differential signal with a high S/N ratio can be obtained from among six differential signals that are obtained from the at least one signal obtained from the at least one second electrode 110B and at least three signals obtained from the at least three first electrodes 110A.

Thus, it is possible to provide an electrocardiogram data detection device 100 that can detect electrocardiogram data of a person sitting on a seat, even when installed in the seat of a vehicle.

The number of second electrodes 110B may be less than the number of first electrodes 110A. A larger number of the first electrodes 110A in the first region 12A where signals can be acquired more stably enables stable acquisition of differential signals and stable detection of electrocardiogram data.

The center of the second region 12B may be located to the upper right of the center of the first region 12A in a up-down and left-right relationship based on the human body of a person sitting on the seat 10. In this case, at least one second electrode 110B is located to the upper right of at least three first electrodes 110A and, therefore, a differential signal with a high S/N ratio can be obtained using the second electrode 110B, enabling highly accurate acquisition of electrocardiogram data.

The at least three first electrodes 110A may be disposed above a height position corresponding to the height position of the iliac crest of a person sitting on the seat 10, and the at least one second electrode 110B may be disposed below a height position corresponding to the height position of the inferior angle of the scapula of the person sitting on the seat 10. Since at least three first electrodes 110A are disposed above the iliac crest, the signals can be stably obtained from a part where there are fewer large bones above the pelvis. Since one second electrode 110B is disposed below the inferior angle of the scapula, the signal can be stably obtained from a part where there are fewer large bones below the scapula. Therefore, the electrocardiogram data detection device 100 can be provided that can stably detect the electrocardiogram data of a person sitting on the seat even when installed in a seat of a vehicle.

While an electrocardiogram data detection device according to an exemplary embodiment of the present disclosure has been described above, the present disclosure is not limited to the specifically disclosed embodiment. Various modifications and changes can be made without departing from the scope of the claims.

With respect to the above-described embodiment, the following additional notes are disclosed:

### Additional Notes

In accordance with an embodiment of the invention, an electrocardiogram data detection device includes a plurality of electrodes provided in a seat back of a seat that includes a seat bottom and the seat back and that is installed in a vehicle, where the plurality of electrodes are disposed to face the human body of a person sitting on the seat, a differential signal generation unit configured to generate a differential signal between signals of two of the plurality of electrodes, where the differential signal generation unit generates a plurality of differential signals from the signals of a plurality of pairs of the electrodes, and an electrocardiogram data detection unit configured to obtain electrocardiogram data based on the plurality of differential signals. The plurality of electrodes include at least three first electrodes disposed in a width direction of the seat back within a first region of the seat back close to the seat bottom and at least one second electrode disposed within a second region of the seat back located above the first region, and the plurality of differential signals are at least six differential signals obtained from the signals of the at least three first electrodes and the signal of at least one second electrode.

In the electrocardiogram data detection device described in the above-mentioned embodiment, the number of the second electrodes may be less than the number of the first electrodes.

In the electrocardiogram data detection device described above, in either case, the center of the second region may be located to the upper right of the center of the first region in an up-down and left-right relationship based on the human body of the person sitting on the seat.

In the electrocardiogram data detection device in any one of or any combination of the above-described cases, the at least three first electrodes may be disposed above a height position corresponding to the height position of the iliac crest of the person sitting on the seat, and the at least one second electrode may be disposed below a height position corresponding to the height position of the inferior angle of the scapula of the person sitting on the seat.

1. An electrocardiogram data detection device installed on a vehicle seat having a seat bottom and a seat back, the electrocardiogram data detection device comprising:
a plurality of electrodes provided in the seat back so as to face a body of a person sitting on the vehicle seat, each electrode outputting a measurement signal;
a differential signal generation unit configured to generate a differential signal between a pair of measurement signals from a corresponding pair the of electrodes, so as to generate a plurality of differential signals by selecting a plurality of pairs from among the plurality of electrodes; and
an electrocardiogram data detection unit configured to obtain electrocardiogram data based on the plurality of differential signals,
wherein the plurality of electrodes include:
at least three first electrodes disposed along a width direction of the seat back in a first region of the seat back; and
at least one second electrode disposed in a second region of the seat back, the first region being closer to the bottom seat than the second region which is located above the first region, and
wherein the plurality of differential signals includes at least six differential signals obtained from the measurement signals of the at least three first electrodes and the measurement signal of at least one second electrode.

2. The electrocardiogram data detection device according to claim 1, wherein a number of the at least one second electrode is smaller than a number of the first electrodes.

3. The electrocardiogram data detection device according to claim 1, wherein a center of the second region is located to an upper right of a center of the first region, up-down and left-right relationships being defined based on the body of the person sitting on the vehicle seat.

4. The electrocardiogram data detection device according to claim 1,
wherein the at least three first electrodes are disposed above a height position corresponding to a height position of an iliac crest of the person sitting on the vehicle seat, and
wherein the at least one second electrode is disposed below a height position corresponding to a height position of an inferior angle of a scapula of the person sitting on the vehicle seat.
